(19) United States
(12) Patent Application Publication
(10) Pub. No.: US 2003/0004495 A1
(43) Pub. Date: Jan. 2, 2003

(54) Apparatus and methods for volumetric CSF removal

(75) Inventor: Tom A. Saul, El Granada, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: EUNOE, INC., Redwood City, CA

(21) Appl. No.: 10/224,046

(22) Filed: Aug. 19, 2002

Related U.S. Application Data
(63) Continuation-in-part of application No. 09/654,967, filed on Sep. 5, 2000.
Application No. 09/654,967 is a continuation of application No. 08/901,023, filed on Jul. 25, 1997, now Pat. No. 6,264,625.
Application No. 08/901,023 is a continuation-in-part of application No. 08/678,191, filed on Jul. 11, 1996, now Pat. No. 5,980,480.
(63) Continuation-in-part of application No. 10/138,082, filed on May 3, 2002.
Application No. 10/138,082 is a continuation of application No. 09/189,037, filed on Nov. 10, 1998, now Pat. No. 6,383,159.

(51) Int. Cl. A61M 1/00

(52) U.S. Cl. 604/540; 604/500; 604/9

(57) ABSTRACT

Apparatus and methods for removing cerebral spinal fluid (CSF) from a CSF space of a patient at constant volumetric rates rely on the intermittent timed or controlled opening of on-off and other control valves. When using valves, the volume of fluid drained may be measured to time the opening and closing of the valves. Alternatively, the valves can be used to control into and/or out of an accumulator, where the accumulator is drained on a periodic timed basis to achieve constant drainage over a one day or other predetermined time period. Such controlled flow volumes may also be achieved using positive displacement pumps which are operated for predetermined time intervals during each one day or other predetermined time period.

### CROSS REFERENCES TO RELATED APPLICATIONS

The present application is a continuation-in-part of U.S. application Ser. No. 09/654,967 (Attorney Docket No. 018050-000120US), filed on Sep. 5, 2000, which was a continuation of U.S. application Ser. No. 08/901,023 (Attorney Docket No. 018050-000110US), filed on Jul. 25, 1997, now U.S. Pat. No. 6,264,625, which was a continuation-in-part of U.S. application Ser. No. 08/678,191 (Attorney Docket No. 018050-000510US), filed on Jul. 11, 1996, now U.S. Pat. No. 5,980,480, the full disclosures of which are incorporated herein by reference. The disclosure of the present application is also a continuation-in-part of U.S. application Ser. No. 10/138,082 (Attorney Docket No. 018050-000510US), filed on May 3, 2002, which was continuation of U.S. application Ser. No. 09/189,037 (Attorney Docket No. 018050-000500US), filed on Nov. 10, 1998, now U.S. Pat. No. 6,383,159. The full disclosures of each of these patents and patent applications are incorporated herein by reference.

### BACKGROUND OF THE INVENTION

Field of The Invention. The present invention relates generally to medical devices and methods. More particularly, the present invention relates to improved devices and methods for removing cerebrospinal fluid (CSF) from the CSF space of a patient to treat Alzheimer's disease and other diseases of the central nervous system (CNS).

Alzheimer's disease is a degenerative brain disorder which is characterized clinically by progressive loss of memory, cognition, reasoning, judgment, and emotional stability and which gradually leads to profound mental deterioration and ultimately death. Alzheimer's disease is the most common cause of progressive mental failure (dementia) in aged humans and is estimated to represent the fourth most common medical cause of death in the United States. Alzheimer's disease has been observed in all races and ethnic groups worldwide and presents a major current and future public health problem. The disease is currently estimated to affect about two to four million individuals in the United States alone and is presently considered to be incurable.

Recently, a promising treatment for Alzheimer's disease has been proposed. The proposed treatment relies on the removal of cerebrospinal fluid (CSF) from the CSF space (which includes the subarachnoid space, the ventricles, the vertebral column, and the brain interstitial space) of a patient suffering from Alzheimer's disease. The treatment is presently believed to be based on the principle that in at least some cases, the characteristic lesions, referred to as senile (or amyloid) plaque and other characteristic lesions in the brain associated with Alzheimer's disease result from the retention of certain toxic substances in the CSF of the patient. A number of suspected pathogenic substances, including toxic, neurotoxic, and pathogenic substances, have been identified to date, including βamyloid peptide (Aβ-42 amyloid), MAP, tau, and the like. It is believed that freshly produced CSF has lower levels or is free of these toxic substances. Thus, it is believed that removal of CSF from the patient's CSF space will reduce the concentration of such substances and significantly forestall the onset and/or progression of Alzheimer's disease and other CNS diseases. The therapeutic effect may also arise from improved transport of other normal substances which may be present at toxic or deleterious concentrations within the CSF, where CSF removal reduces such concentrations. While these mechanisms are believed to be responsible for the therapeutic action, this explanation is intended to help understand such action, and is not intended to limit the scope of the appended claims in any way. This treatment for Alzheimer's disease has recently been described in Rubenstein (1998) The Lancet, 351:283-285, and published PCT application WO 98/02202, which corresponds to parent application Ser. No. 08/901,023.

Hydrocephalus is another condition which is treated by removing CSF from a patient's CSF space, in particular from the cerebral ventricles. Hydrocephalus is characterized by an elevated intracranial pressure resulting from excessive production or retention of CSF, and the removal of CSF has been found to be a highly effective treatment for the condition. Numerous specific catheters and shunts have been designed and produced for the treatment of hydrocephalus, occult hydrocephalus, and other CSF disorders.

The removal of CSF for the treatment of either Alzheimer's disease or hydrocephalus can be accomplished using a wide variety of apparatus which are capable of collecting CSF in the CSF space, preferably from the intracranial ventricles, and transporting the collected fluid to a location outside of the CSF space. Usually, the location will be an internal body location, such as the venous system or the peritoneal cavity, which is capable of harmlessly receiving the fluid and any toxic substances, but it is also possible to externally dispose of the CSF using a transcutaneous device. An exemplary system for removing CSF from a patient's CSF space is illustrated in FIG. 1 and includes an access component 12, a disposal component 14, and a flow control component 16.

While the system of FIG. 1 in general will be suitable for the treatment of both Alzheimer's disease and hydrocephalus, specific characteristics of the flow control component should be quite different because of the different nature of the two diseases. Treatment of hydrocephalus is typically accomplished by a controlled or uncontrolled some threshold value in order to maintain intracranial pressure within normal physiological limits.

A continuous pressure-responsive flow control valve adapted especially for the treatment of Alzheimer's disease patients is described in U.S. Pat. No. 6,383,159. In particular, the CSF removal devices of the '159 patent rely on pressure-compensated CSF removal to achieve a desired generally constant flow rate where a target volume of CSF is removed at a more or less constant flow rate during the day. The valve is designed to provide such continuous flow removal even while the patient's cerebral and ventricular pressures remain at their normal levels.

Such continuous removal of CSF over the course of each day may not always be optimal or even desirable. Even small errors in the desired removal rates may accumulate over time, resulting in excessive volumetric removal of CSF. While the patient's endogenous production of CSF may be able to accommodate any such variations, it would still be desirable to provide CSF drainage catheters which operate on different principles.

For these reasons, it would be desirable to provide apparatus and methods for removing CSF from the CSF space of a patient, where such apparatus and methods could achieve controlled and accurate volumetric removal of the CSF. At least some of these objectives will be met by the invention described hereinafter.

Description of Background Art. The treatment of Alzheimer's disease by removing cerebrospinal fluid from the CSF region of the brain is described in U.S. Pat. Nos. 5,980,480; 6,246,625; and 6,383,159; as well as co-pending U.S. application Ser. Nos. 09/654,967; filed on Sep. 5, 2000; 09/692,593, filed on Oct. 19, 2000; 10/138,082, filed on May 3, 2002; 60/311,307, filed on Aug. 9, 2001; 60/313,938, filed on Aug. 21, 2001; and 60/357,401, filed on Feb. 15, 2002, each of which are assigned to the assignee of the present invention. The full disclosures of each of these patents and applications are incorporated herein by reference.

Methods and shunts for treating hydrocephalus are described in U.S. Pat. Nos. 3,889,687; 3,985,140; 3,913,587; 4,375,816; 4,377,169; 4,385,636; 4,432,853; 4,532,932; 4,540,400; 4,551,128; 4,557,721; 4,576,035; 4,595,390; 4,598,579; 4,601,721; 4,627,832; 4,631,051; 4,675,003; 4,676,772; 4,681,559; 4,705,499; 4,714,458; 4,714,459; 4,769,002; 4,776,838; 4,781,672; 4,787,886; 4,850,955; 4,861,331; 4,867,740; 4,931,039; 4,950,232; 5,039,511; 5,069,663; 5,336,166; 5,368,556; 5 385,541; 5,387,188; 5,437,627; 5,458,606; PCT Publication WO 96/28200; European Publication 421558; 798011; and 798012; French Publication 2 705 574; Swedish Publication 8801516; and SU 1297870. A comparison of the pressure-flow performance of a number of commercially available hydrocephalus shunt devices is presented in Czosnyka et al. (1998) Neurosurgery 42: 327-334. A shunt valve having a three-stage pressure response profile is sold under the Orbis-Sigma® tradename by Nitinol Medical Technologies, Inc. Boston, Mass. 02210 (formerly by Cordis). U.S. Pat. No. 5,334,315, describes treatment of various body fluids, including cerebrospinal fluids, to remove pathogenic substances therefrom.

Articles discussing pressures and other characteristics of CSF in the CSF space include Condon (1986) J. Comput. Assit. Tomogr. 10:784-792; Condon (1987) J. Comput. Assit. Tomogr. 11:203-207; Chapman (1990) Neurosurgery 26:181-189; Magneas (1976) J. Neurosurgery 44:698-705; Langfitt (1975) Neurosurgery 22:302-320.

### BRIEF SUMMARY OF THE INVENTION

Devices and methods according to the present invention provide for the volumetric removal of cerebrospinal fluid (CSF) from the CSF space of a patient. The devices and methods are particularly intended for the treatment of Alzheimer's disease and other conditions which are caused by or otherwise related to the retention and/or excessive accumulation of toxic and other substances in the CSF. In addition to Alzheimer's disease, the present invention will be useful for treating other conditions resulting from the accumulation of toxic substances and resulting lesions in the patient's brain, such as Down's Syndrome, hereditary cerebral hemorrhage with amyloidosis of the Dutch-Type (HCHWA-D), and the like. Other treatable conditions relating to the presence or excessive accumulation of potentially harmful substances include epilepsy, Parkinson's disease, polyneuropathies, multiple sclerosis, amyotrophic lateral sclerosis (ALS), myasthenia gravis, muscular dystrophy, dystrophy myotonic, other myotonic syndromes, polymyositis, dermatomyositis, brain tumors, Guillain-Barre-Syndrome, and the like.

Devices and methods of the present invention are particularly intended for treating conditions in patients having “normal” intracranial pressures, i.e. intracranial pressures below 200 mm H₂O when the patient is reclining and above 170 mm H₂O when the patient is upright (where the pressures are measured relative to the ambient). In contrast, patients suffering from hydrocephalus will have constant or periodic elevated intracranial pressures above 200 mm H₂O (when reclining), often attaining levels two or three times the normal level if untreated. The devices and methods of the present invention are generally not intended for the treatment of patients having chronically elevated intracranial pressures in general and patients suffering from chronic hydrocephalus in particular.

The differences in untreated intracranial and ventricular pressures as well as the different treatment end points (the treatment of hydrocephalus requires lowering of elevated pressures while treatments according to present inventions require lowering of the concentrations of substances in the CSF) require significantly different treatment devices and methods.

By “volumetric removal” it is meant that the methods and apparatus of the present invention will remove a volume of CSF within a target range during a predetermined time period, usually one day (24 hours) rather than in response to intracranial pressure. For the treatment of Alzheimer's disease and other toxic-related conditions, the volume of CSF removed during each one day time period will be in the range from 15 ml to 1500 ml, usually from 40 ml to 300 ml, and more usually from 60 ml to 100 ml. Changes in intracranial pressure resulting from patient posture, positions, or other factors, will have little or no effect on the volume of CSF to be removed.

While the preferred removal ranges for each one-day period have been set forth, it will be appreciated that these volumes could be removed on an hourly, weekly, or other periodic time basis. Moreover, while it will generally be preferred to remove the same volumetric amounts of CSF over successive one-day or other time periods, the present invention also encompasses methods and apparatus for removing different volumes of CSF over successive time periods and/or the removal of identical CSF volumes of different successive time periods. For example, it may be desirable to remove a majority or all of the daily CSF volume during the day when the patient is active, which can be accomplished with the present invention. Alternatively, it might be desirable to remove CSF at night while the patient sleeps, which can also be accomplished with the present invention.

Such volumetric removal may be accomplished in at least several ways. First, the volume of CSF drained over time may be measured and monitored. Once a target volume of CSF has been removed, an on-off or other control valve may be actuated to stop the flow.

Such measurement and control may be performed once per day, or many times per day. In either case, however, the total volume of CSF removed in that day will fall within the above target ranges.

A second exemplary approach can employ a pump together with measurement and monitoring of the amount of CS fluid removed. Starting and stopping of the CSF removal can be accomplished simply by turning off and on the pump. Optionally, valve(s) could also be provided for a more complete shut-off.

Third, the CS fluid could be removed using a positive displacement pump having a flow output controlled by pump speed, and not dependent on patient intracranial pressure. Thus, the target volume of CSF to be removed can be programmed by turning on and off the pump in a predetermined pattern. The pump could be turned on once per day to remove the total desired target volume, or could be actuated numerous times during the day to achieve the same volume.

A fourth approach could use one or more accumulators in combination with one or more on-off valves. By allowing the accumulator to fill and drain in a time-controlled manner, known volume(s) of CSF can be drained during each one-day period. The accumulator could have a blocking valve immediately upstream, in which case the valve would be opened in order to fill the accumulator and be closed after the accumulator is filled. Drainage of the accumulator could be controlled by a second valve. Alternatively, the accumulator could have a flow resistor at its outlet which would permit the accumulator to fill rapidly (the valve would provide a low resistance entrance) while a relatively low percentage of the volume is lost through the flow restrictor. After the valve is closed, the CSF could then drain to the disposal location. The volume of the accumulator and the outlet flow rate would, of course, have to be selected so that there would be sufficient time for drainage of the accumulator before the next cycle of operation was to be initiated.

The accumulator could also have a single one-off valve at its outlet. In that case, the inlet would have to have a relatively high flow resistance. Filling of the accumulator with outlet valve closed would occur over a relatively long period. Once filled, however, the accumulator could be rapidly emptied by opening the outlet valve which would have a very low flow resistance. While the outlet valve was open, flow through the high flow resistance inlet would be relatively low. After drainage, the outlet valve would be closed, allowing the accumulator to once again fill. The next cycle of drainage would then occur according to the predetermined pattern. In all cases, the accumulator will typically have a fill volume in the range from 10⁻³ ml to 40 ml, usually from 0.1 ml to 2 ml, and will be filled and drained from once to 1.5×10⁶ times, usually from 6 to 15,000 times, during each one-day period.

Thus, methods according to the present invention for removing CSF from a patient's subarachnoid space comprise establishing a flow path between the subarachnoid space and a drainage location in the patient's body. Flow through the flow path is then modulated to remove a target volume of CSF within each one-day period. The target volume of CSF to be removed is preferably in the ranges set forth above. Modulating the flow through the flow path may comprise opening an on-off valve. In such case, the desired volume of CSF to be removed may be controlled by measuring the time the valve has been opened and closing the valve after a predetermined period of time has elapsed. Alternatively, the desired CSF volume to be removed may be controlled by measuring the volume of CSF which has been removed over time and closing the valve after a predetermined volume of the fluid has been removed. In either case, the valve may be opened once and closed once during each one-day period, or may be opened and closed multiple times, where the aggregate or total volume removed as a result of each valve opening and closing results in the total removal within the above-described target volume range. When the valve is opened and closed based on time, the time duration will typically be in the range from 1 hour to 8 hours or the flow rate is in the range from 0.5 ml per hour to 40 ml per hour. In some instances, the valve will be opened many times, e.g., from 2 to 10⁸ times, usually from 20 to 10⁵ times, and more usually from 50 to 300 times, during each one-day period. Thus, the volume of CS fluid removed in any single valve opening may vary greatly, typically being from 10⁻⁵ ml to 40 ml, usually from 0.01 ml to 30 ml, and more usually from 0.1 ml to 19 ml, each time the valve is opened. It is also important to control the drainage rate of CSF so that it never exceeds a safe level. Thus, the flow path will be arranged so that the CS fluid removed in any 15-minute period will not exceed 15 ml and in any one hour, will not exceed 50 ml.

Apparatus according to the present invention for removing CSF comprise a conduit comprising a first opening and a second opening. The first opening of the conduit is adapted to be disposed in fluid communication with a space within a patient's subarachnoid space, and the second opening is adapted to be disposed in fluid communication within another portion of the patient's body. A flow rate control device is attached to the conduit between the first and second openings. The flow rate control device is adapted to provide volumetric control of CSF drained through the conduit and may comprise a valve, pump, accumulator, controller, programmable controller, power source, and the like, as discussed in more detail hereinbelow.

For example, when the flow rate control device comprises a valve, the valve may be controlled by a timer or programmable controller. Simply timing the closing and opening of a valve according to a predetermined time schedule will not always provide the degree of accuracy desired. Thus, it is often preferred to control opening and closing of the valve based on the measured volume of CSF which has been drained through the valve. Alternatively, the valve may be opened and closed according to predetermined schedule implemented by a timer, and the accumulator described above utilized to control the total volume of CSF which is drained in any one-day period. The valve will be opened and closed a set number of times during the day, with the time interval(s) of opening and closing being selected to permit filling of the accumulator once during each cycle. By limiting the flow into or out of the accumulator as described above, significant unintended leakage of the CSF from the accumulator can be avoided.

In an additional aspect, the present invention comprises kits, including a ventricular catheter, a peritoneal catheter, and flow rate control module which can be disposed between the ventricular and peritoneal catheters. The flow rate control module will provide for volumetric flow control through the attached catheters. The kit will further comprise instructions for use setting forth any of the methods described hereinabove. The kit may further comprise a package for containing the catheters, the flow rate control module, and the instructions for use. Typical packages include boxes, packages, tubes, pouches, and the like. The catheters and the flow rate control module will typically be maintained sterilely within the packaging.

### BRIEF DESCRIPTION OF THE DRAWINGS

FIG. 1 is a schematic illustration showing the components and placement of a conventional system for removing CSF from a CSF space of the brain.

FIG. 1A is a more detailed view of the CSF space including the brain and the spinal column.

FIG. 2 is a block diagram illustrating a controlled valve system construed in accordance with the principles of the present invention.

FIG. 3 is a block diagram illustrating an accumulator system constructed in accordance with the principles of the present invention.

FIG. 4 is a schematic illustration of a first embodiment of an accumulator system having a controlled outlet valve.

FIG. 5 is a schematic illustration of a second embodiment of an accumulator system having a controlled outlet valve.

FIG. 5A shows a pump which may be used as the flow rate control device in the present invention.

FIG. 5B shows a screw pump which may be used as the flow rate control device of the present invention.

FIG. 6 illustrates a kit according by the present invention

### DETAILED DESCRIPTION OF THE INVENTION

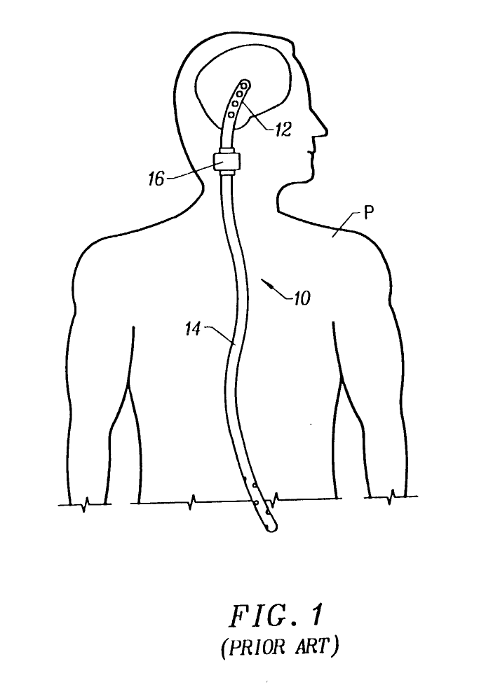

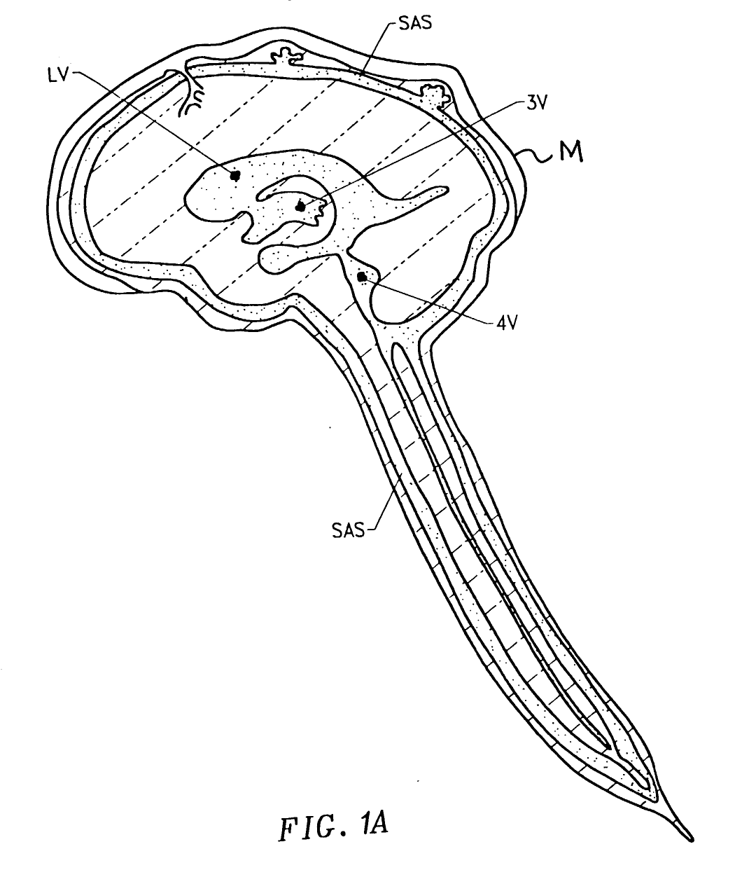

The brain and spinal cord are bathed in cerebrospinal fluid (CSF) and encased within the cranium and vertebral column inside a thin membrane known as the meninges (FIG. 1A). The space within the meninges M, which is the three-membrane complex enveloping the brain and spinal cord, consists of the subarachnoid space SAS, including the ventricles (including the lateral ventricle LV, third ventricle 3V, and fourth ventricle 4V), the vertebral column, and the brain interstitial spaces. The total space within the meninges M is referred to herein as the “CSF space.” The volume of the brain intracranial spaces is on average about 1700 ml. The volume of the brain is approximately 1400 ml, and the volume of the intracranial blood is approximately 150 ml. The remaining 150 ml is filled with CSF (this volume will typically vary within 60 ml to 290 ml). The CSF circulates within the CSF space. CSF is formed principally by the choroid plexuses, which secrete about 80% of the total volume of the CSF. The sources of the remainder are the vasculature of the subependymal regions, and the pia matter. The total volume of the CSF is renewed several times per day, so that about 500 ml are produced every 24 hours (equivalent to about 20 ml/hr or 0.35 ml/min) in healthy adults. The production rate varies in the old and the young.

The cerebrospinal fluid is absorbed through the arachnoid villi, located principally over the superior surfaces of the cerebral hemispheres. Some villi also exist at the base of the brain and along the roots of the spinal nerves. The absorptive processes include bulk transport of large molecules and as well as diffusion across porous membranes of small molecules. The production and absorption of CSF are well described in the medical literature. See, e.g., Adams et al. (1989) “Principles of Neurology,” pp. 501-502.

While CSF is naturally absorbed and removed from circulation, as just described, it is presently believed that certain toxic or other substances may be present in the CSF, such as those associated with Alzheimer's disease, and may accumulate or persist to an extent which can cause Alzheimer's disease or other disorders. Such substances are either produced in excess, removed at a rate slower than their production rate, or not properly circulated so that they accumulate or stagnate and increase in toxicity and/or reach a threshold concentration in which they become toxic in the brain or elsewhere within CSF space.

The present invention is directed at particular devices and methods for the improved circulation of CSF and/or removal of such substances from the CSF in order to treat, inhibit, or ameliorate conditions associated with such toxic and other substances. In particular, the present invention is directed at reducing the concentration of such substances in CSF by removing portions of the CSF from the CSF space. Such removal is believed to either enhance production of the CSF and/or enhance circulation of the CSF while assuring that the total volume of CSF in the CSF space is not reduced below a safe level. Moreover, the rates at which the CSF is removed are generally quite low (when compared to the rates of removal for treatment of the hydrocephalus) so that the likelihood of removing excessive amounts of CSF is very low.

By removing CSF from the CSF space, the toxic substances present in the removed CSF will thus be removed from the CSF space and will not be available for absorption or recirculation. So long as the rate of removal exceeds the rate of production of such substances, the concentration of such substances can be reduced. Usually, the removed CSF will be directed to a natural disposal site within the patient's body which can tolerate the toxic substance. Suitable sites, particularly for those substances associated with Alzheimer's disease as discussed above, include the venous system, peritoneal cavity, the pleural cavity, and the like. In the event that a toxic substance would be deleterious if transferred within the patient's body, or for any other reason, it is also possible to remove the CSF from the patient's body, e.g. using a transcutaneous catheter and external collection bag or other receptacle. It will generally be preferable to maintain the entire system subcutaneously for patient convenience and to reduce the risk of infection.

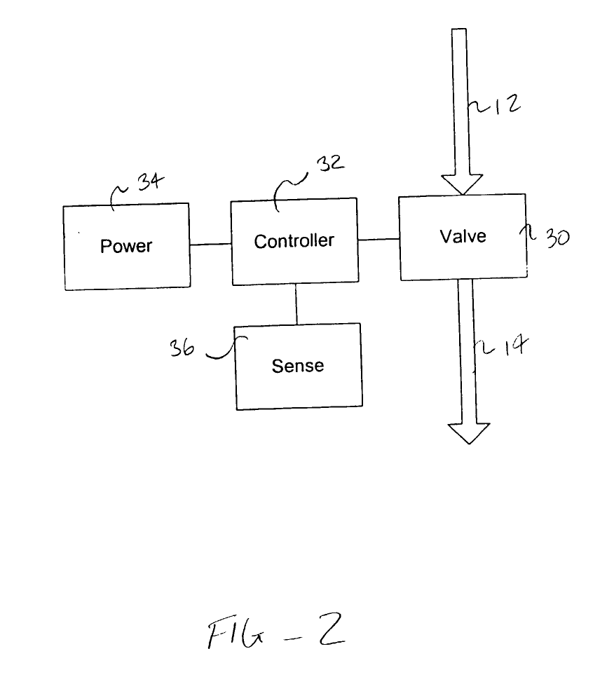

Referring now to FIG. 2, a first control system and protocol for performing the methods of the present invention will be described. An on-off or other flow control valve 30 is provided between a ventricular catheter 12 and a peritoneal catheter 14, which may be essentially identical to those described in connection with FIG. 1 above. The valve 30 will be turned on and off or modulated by a controller or actuator 32 which will have a power source 34. The power source may comprise a mechanical energy source, such as a spring, bellows, or the like, or more likely will comprise an electrical energy storage device, typically a chemical battery. In the latter case, the electrical energy storage device will preferably be rechargeable using external RF energy, optical energy, or the like. In the case of mechanical power sources, they may be recharged by patient motion, or the like.

The controller 32 is meant to be any instrument which utilizes power from source 34 to turn on and off or otherwise modulate the valve 30. The controller may further comprise control circuiting, timing circuity, sensing circuitry, and the like to permit programmed or otherwise controlled operation of the valve 30. For the most part, it will be desirable to turn the valve on and off to permit a controlled volumetric drainage of the CSF. Such valve operation may be in response to a predetermined time schedule but will more effectively be in response to the measured drainage of the CSF during any period the valve is open.

When CSF drainage is being controlled based on volume, it will be necessary to sense the volume of flow using a sensing device 36. The sensing device will preferably totalize flow through the valve, and the controller 32 will turn on and off or otherwise modulate the valve flow periodically based on the total volumetric flow observed over time. Most simply, the valve could be opened once a day (based on a timer present in the controller 32 or sensor 36) and then closed after the sensor 36 has determined that the target volume has been drained. Such an approach would be effective so long as the maximum 15-minute and hourly depletion volumes described above are not exceeded. In other cases, it might be desirable to open and close the valve more than once during each one-day period, possibly opening and closing the valve up to 2×10⁸ times as described above, or usually, the valve would be opened 10⁵ times or fewer, usually 300 times or fewer, and preferably 50 times or fewer.

When electrically powered controllers and sensors are employed, the valve will also typically be electrically controlled. Suitable electrically controlled valves are well described in patent and technical literature. Alternatively, mechanically controlled valves are described in U.S. Pat. No. 6,264,625, the full disclosure of which has previously been incorporated herein by reference.

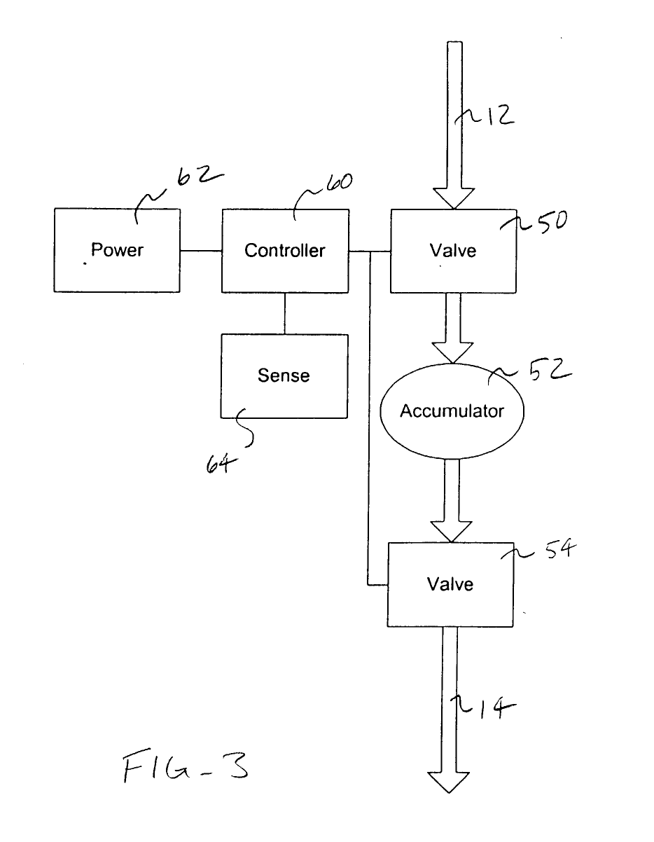

A CSF drainage system using an accumulator to measure the volumetric drainage is schematically illustrated in FIG. 3. The system of FIG. 3 will include at least one valve 50, an accumulator 52, and optionally a second valve 54 which may further optionally be used in place of the first valve 50, as described in more detail below. The system of FIG. 3 will also include a controller 60 for operating the valve 50 (and alternatively or additionally the valve 54), a power source 62, and optionally a sensor 64.

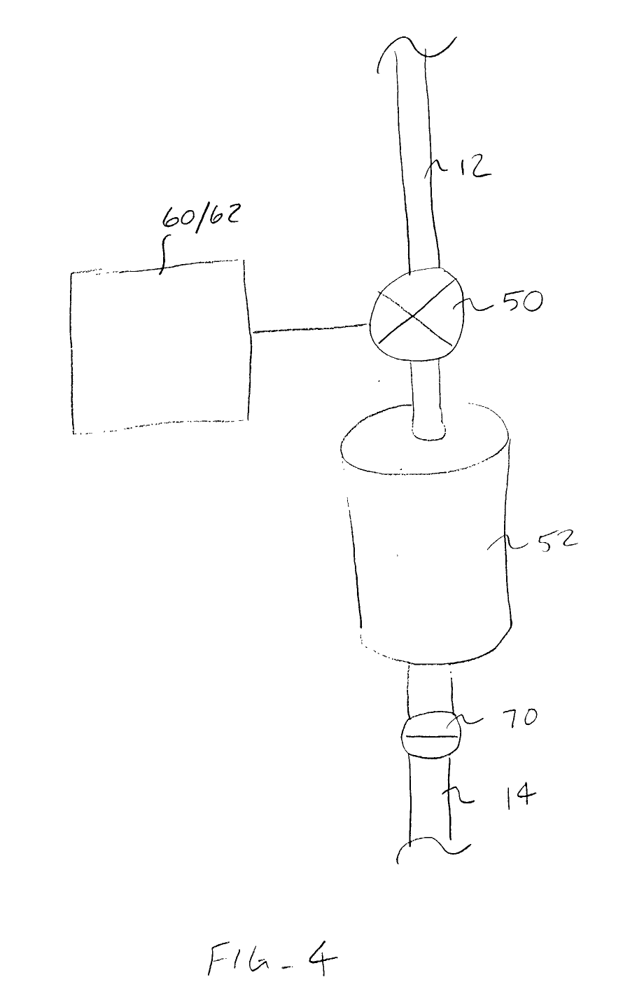

A first example of a system employing an accumulator 52 is shown in FIG. 4. Ventricular catheter 12 is connected to an on-off control valve 50 which is connected to a combined power supply and controller 60/62. The accumulator has a volume in the ranges set forth above, and is attached to the peritoneal catheter 14 through a flow restrictor 70. The flow restrictor 70 provides a flow resistance which greatly inhibits the out flow of CSF from the accumulator while the inlet valve 50 is open. Thus, the accumulator can be filled by opening valve 50 based on a signal from the controller/power supply 60/62. The signal can be provided based on a timer included within the controller 60, e.g., once per one-day period. The valve 50 will remain open for a time which is more than sufficient to fill the accumulator 52. It will be appreciated that, once the accumulator 52 is filled, flow into the accumulator will essentially stop, although a small amount of leakage will continue through the flow restrictor 70. After sufficient time has passed for the accumulator to be filled, the valve 50 will be closed, and the accumulator 52 allowed to drain over time through the flow restrictor 70. The cycle can then begin again, typically 24 hours or other fixed time interval later, after the accumulator 52 has completely drained. In this way, a very precise volume of CSF can be drained each one-day period. Of course, it would be possible to actuate valve 50 to perform two, three, four, or more cycles in any one-day period.

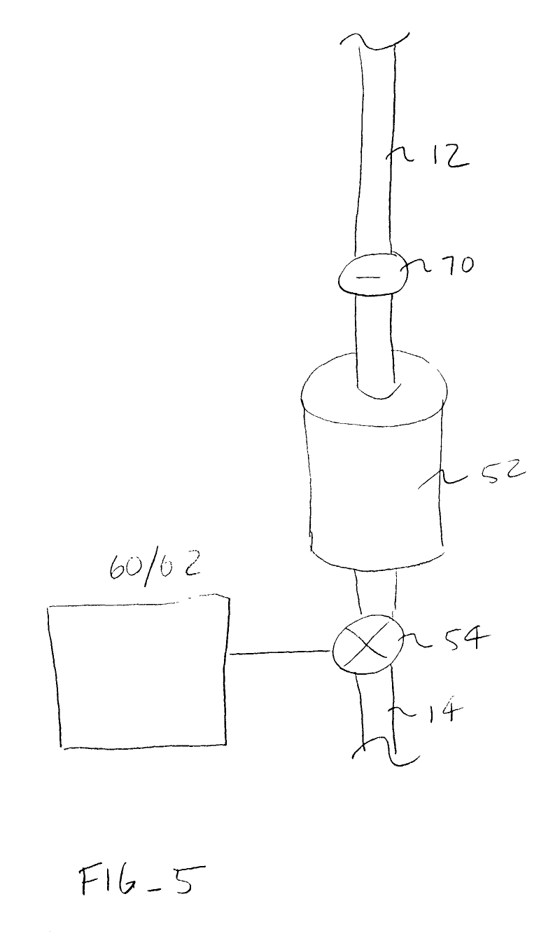

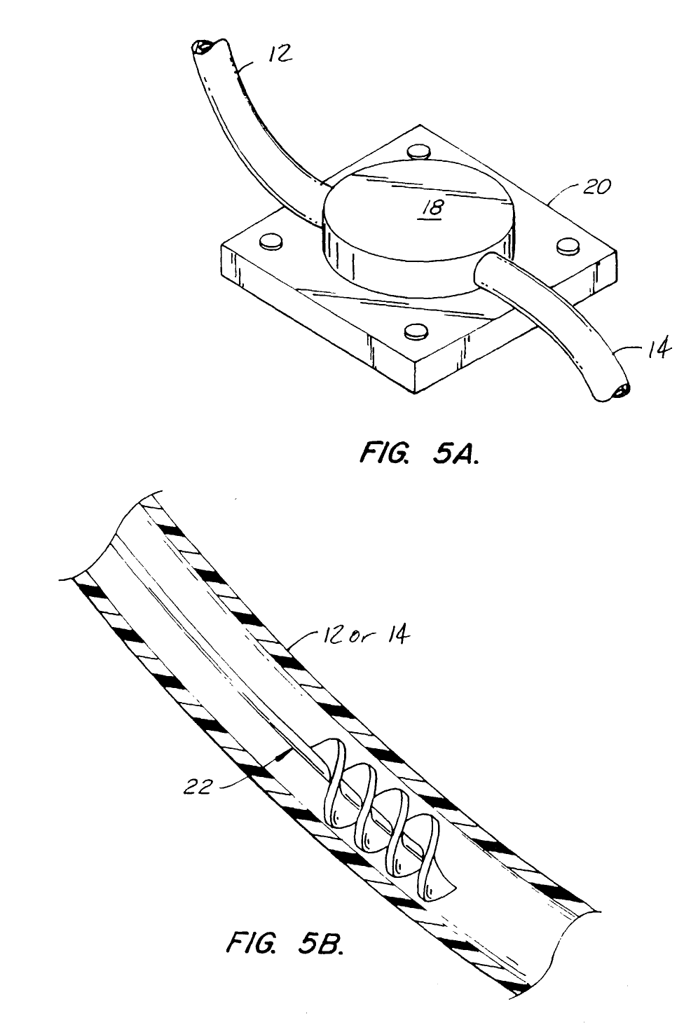

A second specific example of the accumulator system of FIG. 3 is illustrated in FIG. 5. In the system of FIG. 5, the controller/power supply 60/62 is connected to drive the second on-off valve 54. The accumulator 52 fills from ventricular catheter 12 through the flow restrictive element 70. While the valve 54 is closed, the accumulator will slowly fill with flow essentially stopping after the accumulator has completely filled. After the accumulator is filled, the controller/power supply 60/62 can open the valve 54 which will permit rapid drainage of the accumulator 52. Of course, a small amount of CSF will drain through the flow restrictor 70, but such leakage will be very small when compared to the volume of CSF released from the accumulator 52. After sufficient time has passed to permit complete emptying of the accumulator, the valve 54 will be closed, and filling of the accumulator will begin again. Such cycles of filling and draining can be performed once each one-day period, or multiple times depending on the precise target volume, volume of the accumulator, and the like.

The system of FIG. 3 can of course accomplish even more accurate measurement of the drained CSF using a pair of valves as illustrated in FIG. 3. In such case, the accumulator may be filled by opening valve 50 while valve 54 remains closed. The accumulator will fill entirely and may be left filled until it is desired to drain the accumulator. At that time, the valve 50 should be closed, and valve 54 opened to permit a rapid draining of the accumulator. After a sufficient time has been allowed for permitting drainage, or drainage of the accumulator is confirmed using the sensor 64, the valve 54 may be closed and valve 50 reopened to permit filling of the accumulator. As the filling and drainage of the accumulator 52 are precisely controlled by the valves 50 and 54, there will be no leakage as with the embodiments of FIGS. 4 and 5. The system of FIG. 3 will, however, will require greater power consumption to operate two valves.

FIG. 5A shows an embodiment in which the fluid flow rate control device is an implantable pump 18 attached between ventricular catheter 12 and peritoneal catheter 14. Pump 18 may be diaphragm pump, piston pump, rotor pump, peristaltic pump, screw pump, or any other suitable pump. The power source for pump 18 may be a battery or other energy storage device, such as a mechanical flywheel with self-winding operation. The pump also may be remotely operated as is known in the art. Pump 19 further may be operated continuously or periodically, either on demand or according to a schedule or program. Pump may be mounted on a baseplate 20 which is adapted for attachment to a port of the patient's anatomy. FIG. 5B illustrates a conventional screw pump arrangement where a screw shaft 22 is mounted for rotation within the ventricular catheter 12 and/or peritoneal catheter 14. The drive may be positioned in a hermetically sealed package mounted to the conduit exterior and arranged within the thorax or peritoneum. The drive may be coupled to screw shaft 22 with a gear transmission as would be apparent to one of ordinary skill in the art. Other screw pump configurations also can be used such as those disclosed in U.S. Pat. No. 4,857,046 to Stevens et al. to U.S. Pat. No. 5,372,573 to Habib.

Such positive displacement pumps will drain a known volume of CSF based on each revolution, cycle, or the like. Thus, the total drained volume in any one-day period can be provided by operating the pump for a predetermined time at a predetermined rate. It is unnecessary to measure the flow or use an accumulator, although measured confirmation of flow might be valuable. It would also be possible to turn the pump off and on or otherwise control the volume delivered based on the measured flow using conventional feedback control algorithms implemented by the controller.

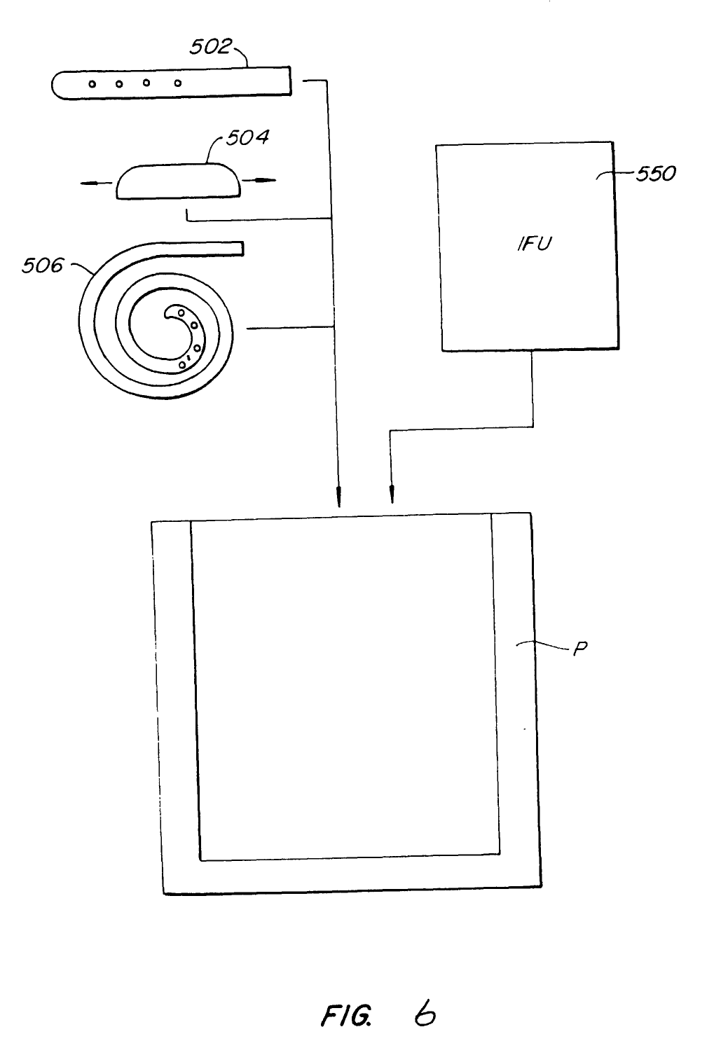

Systems according to the present invention may be provided in a kit form, as illustrated in FIG. 6. The kit will include the system components, such as ventricular access catheter 502, a flow control module 504, and a peritoneal catheter 506, together with instructions for use 550. The instructions for use 550 may set forth any of the methods described in the present application, including methods for implanting the system components within a patient so that the ventricular catheter is at the subarachnoid space, the flow control module is within the thoracic cavity, and the peritoneal catheter terminates within the peritoneum.

The system components and instructions for use will be provided within a package P, which may be in the form of a pouch, box, tray, tube, or other conventional medical package. The instructions for use 550 may be packaged within the package or may be printed on the package, or both. Usually, the system components will be sterilized within the package so they may be used without further sterilization.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for removing cerebrospinal fluid (CSF) from a patient's subarachnoid space, said method comprising:
establishing a flow path between the subarachnoid space and a drainage location in the patient's body; and
modulating flow through the flow path to remove a target volume of CSF within a predetermined time period.

2. A method as in claim 1, wherein the predetermined time period is one day.

3. A method as in claim 2, wherein the target volume is the same for each one-day time period.

4. A method as in claim 1, wherein modulating comprises removing different target volumes of CSF in at least some successive predetermined time periods.

5. A method as in claim 1, wherein modulating comprises removing target volumes of CSF in predetermined time periods having different lengths.

6. A method as in claim 3, wherein the target volume of CSF to be removed in the one-day time period is in the range from 15 ml to 1500 ml.

7. A method as in any of claims 1-6, wherein modulating flow through the flow path comprises opening an on-off valve disposed in the flow path.

8. A method as in claim 7, further comprising measuring the time the valve has been opened and closing the valve after a preselected period of time has elapsed.

9. A method as in claim 7, further comprising measuring the volume of CSF which has been removed and closing the valve after a predetermined volume of removed fluid has been measured.

10. A method as in claim 7, wherein the valve is opened and closed once during each predetermined time period.

11. A method as in any of claims 2-6, wherein the valve is opened for a period in the range from 1 hour to 8 hours and provides a flow rate from 0.5 ml/hour to 40 ml/hour.

12. A method as in claim 7, wherein the valve is opened from 2 to 10⁸ times each predetermined period.

13. A method as in claim 12, wherein the valve is opened for a preselected time sufficient to drain a volume of CSF in the range from 10^{31 5} to 40 ml while the valve is open.

14. A method as in claim 7, wherein the flow path is arranged so that the CSF volume removed in any 15-minute period will not exceed 15 ml and in any one-hour period will not exceed 50 ml.

15. A method as in any of claims 1-6, wherein modulating flow through the flow path comprises filling an accumulator with a predetermined volume of CSF and draining CSF from the accumulator after the accumulator has been filled.

16. A method as in claim 15, wherein the accumulator has a predetermined fill volume in the range from 10⁻³ to 40 ml and the accumulator is filled and drained from 1 time to 1.5×10⁶ times during each one-day period.

17. An apparatus for removing cerebrospinal fluid (CSF) comprising:
a conduit comprising a first opening and a second opening, the first opening of the conduit being adapted to be disposed in fluid communication with a space within a patient's subarachnoid space the second opening being adapted to be disposed in fluid communication with another portion of the patient's body; and
a flow rate control device attached to the conduit between the first and second openings.

18. The apparatus of claim 17, wherein the flow rate control device comprises a valve disposed in the conduit.

19. The apparatus of claim 18, further comprising means for actuating the valve at regular intervals.

20. An apparatus as in claim 17, wherein the flow rate control device comprises at least one valve and at least one accumulator in series with the valve.

21. An apparatus as in claim 20, wherein the valve is disposed upstream of the accumulator, wherein when the valve is open, the accumulator fills with CSF from the first opening much more rapidly than it drains CSF through the second opening.

22. An apparatus as in claim 21, further comprising a controller for opening and closing the valve a predetermined number of times in successive predetermined time periods, wherein the valve is opened for time intervals much longer than the time intervals during which the valve is closed to assure filling of the accumulator each time before it is drained.

23. An apparatus as in claim 22, wherein the controller opens and closes the valve from 1 to 1.5×10⁶ times during each one-day predetermined time period.

24. An apparatus as in claim 20, wherein the valve is disposed downstream of the accumulator, wherein when the valve is open, the accumulator drains CSF through the second opening much more rapidly than it fills from the first opening.

25. An apparatus as in claim 24, further comprising a controller for opening and closing the valve a predetermined number of times in successive predetermined time periods, wherein the valve is opened for time intervals much shorter than the time intervals during which the valve is closed to assure filling of the accumulator each time before it is drained.

26. An apparatus as in claim 25, wherein the controller opens and closes the valve from 1 to 1.5×10⁶ times during each one day predetermined time.

27. An apparatus as in claims 20-25, or 26, wherein the accumulator has a fill volume in the range from 10⁻³ ml to 40 ml.

28. An apparatus as in claim 27, further comprising a controller for opening and closing the valve according to a predetermined time schedule which fills and drains the accumulator so that a volume of CSF in the range from 12 ml to 1500 ml is removed during successive predetermined time periods.

29. An apparatus as in claim 17, wherein the flow rate control device comprises a pump.

30. An apparatus as in claim 29, wherein the pump is a positive displacement pump and wherein the apparatus further comprises a controller for driving the pump at one or more intervals during each one-day successive predetermined time period to drain a target volume of CSF in the range from 12 ml to 1500 ml.

31. An apparatus as in claim 30, wherein the controller is programmable to allow selection of different target volumes within the range.

32. An apparatus as in claim 18, further comprising a flow sensor, an actuator for modulating CSF flow through the valve, and a controller which adjusts the actuator in response to total flow measured by the flow sensor, wherein from 15 ml to 1200 ml of CSF is removed in each one-day predetermined time period.

33. An apparatus as in claim 32, wherein the controller is programmable to allow selection of different target volumes within the range.

34. A kit comprising:
a ventricular catheter;
a peritoneal catheter;
a flow rate control module; and
instructions for use setting forth a method according to claim 1.

35. A kit of claim 34, further comprising a package which contains the catheters, the flow rate module, and the instruction for use.

36. An apparatus for removing cerebrospinal fluid (CSF) from a patient's subarachnoid space, said apparatus comprising:
a conduit comprising a first opening and a second opening, the first opening of the conduit being adapted to be disposed in fluid communication with a space within a patient's subarachnoid space and the second opening being adapted to be disposed in fluid communication with another portion of the patient's body;
an on-off valve disposed to control flow through the conduit between the first and second openings; and
means for turning the valve on and off in response to the volume of CSF which has been removed and/or the time which the valve has been opened.

37. An apparatus as in claim 22, wherein the turning means comprises a timer which opens and closed the valve according to a predetermined schedule.

38. An apparatus as in claim 37, further comprising an accumulator in series with the on-off value, wherein the valve is turned on and off at intervals which assure that the accumulator will be filled without significant drainage only once during each interval.

39. An apparatus as in claim 36, wherein the turning means comprises a flow sensor which measures total flow volume through the conduit while the valve is opened.

40. An apparatus as in claim 39, wherein the turning means further comprises a timer which periodically opens the on-off valve, wherein the valve is closed after a predetermined volume of CSF has been removed while the valve was opened.
